(19) United States
(12) Patent Application Publication
(10) Pub. No.: US 2016/0029883 A1
(43) Pub. Date: Feb. 4, 2016

(54) EYE TRACKING CALIBRATION

(71) Applicant: EYE TRACKING ANALYSTS LTD, Stafford (GB)

(72) Inventor: John Stephen Cox, Stafford (GB)

(21) Appl. No.: 14/780,427

(22) PCT Filed: Mar. 28, 2014
(86) PCT No.: PCT/GB2014/050999

§ 371 Date: Sep. 25, 2015

(30) Foreign Application Priority Data
Mar. 28, 2013 (GB) 1305726.0
Jul. 18, 2013 (GB) 1312873.1

(51) Int. Cl. A61B 3/00; G06F 3/01; G06K 9/00; A61B 3/113

(52) U.S. Cl. CPC A61B 3/0025; A61B 3/113; G06F 3/013; G06K 9/00617; G06K 9/00604

(57) ABSTRACT

Method, system and apparatus for calibrating an eye tracker comprising presenting a subject with a visual target. Detemiining a physical arrangement in space of the subject. Obtaining an eye measurement of the subject. Storing a record of the subject's physical arrangement in space and data derived from the eye measurement of the subject, associated with the visual target presented to the subject. Repeating for one or more further different visual targets.

### FIELD OF THE INVENTION

The present invention relates to a method and system for tracking the gaze or point of regard of a subject and in particular to the calibration of such eye-gaze or tracking systems.

### BACKGROUND OF THE INVENTION

Eye-tracking is a topic of growing interest in the computer vision community. This is largely due to the wide range of potential applications. An eye-tracker device consists of sensors to make eye-gaze measurements and algorithmic techniques to map these eye-gaze measurements into real-world Cartesian space. Such eye-tracker devices can be used in a number of fields such as natural user interfaces for computerised systems, marketing research to assess customer engagement with visual marketing material, software usability studies, assessing product placement in supermarkets, attention monitoring in critical systems, attention monitoring within vehicles and in assistive and augmented communication devices for people with severe motor disabilities.

Eye tracking systems exist in a variety of different forms. There are invasive methods that require the tracking subject to wear special apparatus such as eye-glasses hosting photodetector arrays, contact lenses with sensors or electrodes to be placed on the skin. As these methods can be uncomfortable for the tracking subject, there has been growing interest in non-invasive video-camera based approaches. Such methods employ one or more video cameras located so as to observe the tracking subject's face. The tracking subject's eyes are then located in the video image and the movement of the iris and/or pupil is tracked.

Non-invasive video based eye-tracking methods can be categorised into two distinct groups: interpolation methods and geometry-based methods. Interpolation methods typically use linear or quadratic polynomial models to represent the spatial relationship between image features and gaze. The polynomial functions used in these models depend upon unknown coefficients determined by calibration. Although interpolation methods are quite simple, the limited degree of control over polynomial models and the relatively high degree of system errors is a significant disadvantage. Because the coefficients in the polynomial model are determined during calibration, movement of the tracking subject from the calibration position can make the gaze-estimation very inaccurate.

Geometry-based methods attempt to provide gaze estimation that is tolerant to head movement, and are based on mathematical principles and geometric models. These methods typically require user calibration for gaze estimation purposes. These methods employ 3D eye models and calibrated camera, light sources, and camera positions to estimate line of sight or gaze. Some studies have been carried out in non-calibrated scenarios using projective plane properties for point of regard estimation.

Both the interpolative and geometric based approaches suffer from the problem that iris/pupil tracking necessitates the recording of movements of the iris/pupil within a 2D video image. Such readings will be relative to the local 2D co-ordinate system of the video image. For example, the system might record lateral and longitudinal displacement of the iris/pupil with respect to the centre of the eye or pupil displacement with respect to the glint produced by infra-red illuminators (Purkinje images). These measurements would then be expressed in units specific to the video image. Any variation in the position or orientation of the camera or the tracking subject's head would alter the eye-tracking readings. This causes problems when trying to map eye-gaze readings onto a display screen which exists in the 3D co-ordinate system in which the eye-tracking is taking place.

Interpolative methods use a calibration procedure to map eye-gaze readings onto the display screen. A typical calibration approach involves asking the tracking subject to follow a moving token with his/her eyes as it moves around the display. Doing this allows the system to produce the necessary mapping between eye-gaze measurements and the extents of the display.

However, such calibration methods have a number of drawbacks. Firstly, once the calibration procedure has finished, the tracking subject must remain virtually still with only limited head movements being allowed. This is because the mapping produced by the calibration procedure is between the local 2D co-ordinates of the eye-gaze readings and the extents of the display screen. Any change in the eye-gaze readings (such as occur when the tracking subject moves toward or away from the camera, moves laterally or rotates his/her head) will change the eye-gaze to screen mappings. When this happens, the tracking subject must repeat the calibration procedure so as to create an up-to-date eye-gaze to display mapping.

Another disadvantage of using an overt calibration procedure is that the tracking subject becomes aware that their eyes are being tracked. In situations where eye-tracking is being used as part of a research experiment such as in marketing research, the fact that the tracking subject is aware that his/her eyes are being tracked might influence his/her eye-gaze patterns and bias the results—a phenomenon known as the Hawthorn effect whereby respondents change their behaviour because they know they are being studied.

Therefore, there is required a system and method that overcome these problems.

### SUMMARY OF THE INVENTION

An eye tracking system is calibrated by presenting a subject with a target (on a screen, for example). The subject is asked to look at the target. The location, orientation and position of the subject is determined or measured. The target location is also known and presented at a predetermined known position. The subject's eyes are also measured whilst they are looking at the target. These measurements or data derived from these measurements are stored together with information identifying or characterising the target. Position may also be tracked without tracking orientation.

When the subject (or another subject) is measured with the same or similar location and orientation and with their eye or eyes having the same or similar measurements then it may be assumed that the subject is looking at the same point or position or in the same direction as they did (or when the calibration subject did) when looking at the target. Accuracy may improve as the calibration data set is built further. Therefore, the gaze of new subjects (or the same subject) may be determined without having to present them with calibration targets at the time of making eye-gaze measurements. The system may also stay calibrated following movement of the tracking subject to a new position. Preferably, the stored calibration record corresponds well or is at least is a close match with the subject. The required closeness of the match may be predetermined or be within a set tolerance. However, when there isn't a close match then the stored records may still be used but may require further processing. This may include interpolation or estimation techniques, for example. The eye measurements may be stored as parameters of a function, for example. The function may provide an output that indicates the gaze of the subject for any input eye measurement (not necessarily one that has already been measured).

In accordance with a first aspect there is provided a method for calibrating an eye tracker, the method comprising the steps of:
(a) presenting a subject with a visual target;
(b) determining a physical arrangement in space of the subject;
(c) obtaining an eye measurement of the subject;
(d) storing a record of the subject's physical arrangement in space and data derived from the eye measurement of the subject, associated with the visual target presented to the subject; and

(e) repeating steps a) to d) for one or more further different visual targets. Therefore, the record or calibration store can be added to in order to improve tracking accuracy and reduce the need for further calibration for individual users. The visual target may be a visual calibration target, for example. In other words, the visual target may be an item (e.g. part or whole of an image) with a specific purpose to draw a subject's gaze (e.g. star, dot, cross, etc.) However, the visual target may instead be an item (e.g. part or whole of an image) that has another or primary use. For example, the visual target may be an icon, box, selectable button with a known location that may also draw the gaze of the subject. ‘Pupil Centre Corneal Reflection’ (PCCR) eye tracking is an example technique that requires calibration although there are others. The method may be repeated for one or more additional or new subjects. The physical arrangement in space may be obtained by directly or indirectly measuring the subject (measuring the physical arrangement in space) or by otherwise retrieving measurement data. This may be in real time or from a recording, for example.

The present technique is based upon making eye-measurements and recording the user's physical arrangement in space for a given calibration token.

For example we may do the following:

1) We measure the user's physical arrangement in space

2) We display a calibration token

3) We make eye-measurements

4) We repeat steps 2-3 for each physical arrangement in space.

5) We calibrate for each physical arrangement in space.

However, it may be that some embodiments of the invention may create a mathematical model for the eye and determine the ‘eye-measurements’ from calculations based upon the mathematical model.

For instance, if the ‘eye-measurements’ are based upon the pupil-centre and the centre of one or more Purkinje images, then an embodiment may use a geometric model of the eye to determine the ‘eye measurements’. Hence, the position of the pupil centre and the position of the Purkinje image (glint produced by infra-red illuminator) may be determined by calculation based upon a mathematical model of the eye.

Preferably, the method may further comprise the step of repeating steps a) to e) for one or more different subject physical arrangements in space. The repetition of the steps for different subject physical arrangements in space may be for the same subject after they have moved or altered their physical arrangement in space (e.g. turned, twisted, rotated, or laterally translated) or for more than one different subject.

Optionally, the method may further comprise the step of repeating steps a) to e) for the same subject physical arrangement in space. Therefore, a store of multiple records may be built for the same or different targets with the subject having the same physical arrangement.

Optionally, the data representing the subject's eye measurement is stored as a template representing the eye or eyes of the subject. This template may be simplified representation, for example. Eye measurements may be stored as a template, which can be an image of the eye or eyes. Preferably, the templates may be images.

Optionally, the method may further comprise the step of:

generating parameters of a function having an output that describes the gaze of a subject when provided with an input representing an eye measurement of the subject. The function accepts an input from any subject (e.g. the first second or other subject).

Preferably, the data derived from the eye measurements may be the parameters of the function. Therefore, either the eye measurements may be stored directly or stored as parameters of a function that can accept a new eye measurement and return an indication of the gaze of the subject even when that particular new eye measurement has not been encountered or recorded before.

Optionally, the output may be in the form of coordinates on a field of view. The output may also correspond with a position on a screen that the subject is viewing or a position in space, for example. The gaze may include what the subject is looking at, what direction they are looking in, the distance from the subject that they are viewing, or other information. The gaze may vary and may be continually monitored or determined at a particular instant.

Optionally, the parameters may be generated by interpolating between the locations of the visual targets.

Optionally, the location of the visual targets are coordinates on a field of view, the coordinates corresponding to a gaze of the subject when looking at the visual target.

Advantageously, the function may be a polynomial or a bi-linear function and the parameters are coefficients of the polynomial or bi-linear function.

According to a second aspect, there is provided a method for tracking the gaze of a subject comprising the steps of:

(a) determining a physical arrangement in space of a first subject;

(b) obtaining an eye measurement of the first subject;

(c) matching the determined physical arrangement in space of the first subject with a physical arrangement in space of a second subject presented with one or more visual targets, wherein the physical arrangement in space of the second subject is associated with data derived from one or more eye measurements obtained when the second subject was presented with the one or more visual targets; and

(d) providing an output indicating a gaze of the first subject based on the obtained eye measurement of the first subject and the data derived from the one or more eye measurements of the second subject. Therefore, the gaze of a subject may be monitored or tracked based on previously recorded calibration records for the same or one or more different subjects. The retrieved stored record may be from a different subject or otherwise generated.

Preferably, the output may indicate the gaze of a subject viewing the previously presented visual target. The gaze may include what the subject is looking at, what direction they are looking in, the distance from the subject that they are viewing, or other information. The gaze may vary and may be continually monitored or determined at a particular instant.

Preferably, the output may indicate the direction of gaze of the subject. The output may also correspond with a position on a screen that the subject is viewing or a position in space, for example.

Optionally, the first subject may be the same as the second subject or a different subject.

Optionally, a match between the determined physical arrangement in space of the first subject and the second subject may occur within a matching threshold or according to other criteria. The determined physical arrangement of the subject may not be exactly the same as that of the stored record. Therefore, the match may be made based on a predetermined threshold or threshold. In other words, the match may not need to be perfect but within one or more limits. For example, all records below a matching threshold may be retrieved for a particular obtained physical arrangement.

Preferably, the data derived from the one or more eye measurements of the second subject may be parameters of a function having an output that describes the gaze of a subject when provided with an input representing an eye measurement of the first subject.

Optionally, the output may be in the form of coordinates on a field of view.

Optionally, there may be a plurality of visual targets and the parameters may be generated by interpolating between the locations of the plurality of visual targets.

Optionally, the locations of the visual targets may be coordinates on a field of view or screen, the coordinates corresponding to a gaze of the subject (first, second or other subject) when looking at the visual target.

Optionally, the function may be a polynomial or a bi-linear function and the parameters are coefficients of the polynomial or of the bi-linear function.

Optionally, the matching step may further comprise determining a closest match between the determined physical arrangement in space of the first subject and the physical location in space of the second subject. The determined physical arrangement of the subject and/or obtained eye measurement may not be exactly the same as that of the stored record. Therefore, the match may be made based on a predetermined threshold or threshold. In other words, the match may not need to be perfect but within one or more limits.

Optionally, the matching step may further comprise determining a minimum Euclidian distance between the determined physical arrangement in space of the first subject and a physical arrangement in space of the second subject within any one or more stored records. Other matching methods and algorithms may be used.

Optionally, data derived from the one or more eye measurements of the second subject may comprise a template representing the eyes of the second subject.

Optionally, the step of providing the output may further comprise the steps of:

matching the obtained eye measurement of the first subject with an eye measurement of the second subject obtained when the second subject was presented with a visual target; and

indicating the gaze of the first subject as the location of the visual target.

Optionally, determining the physical arrangement in space of the subject (first, second or any other subject) may further comprise determining an eye reference point from an image of the subject obtained by a camera. The camera may be a still or video camera, for example.

Optionally, the method may further comprise determining a distance Li, which is a distance, as recorded in a camera image, between facial features of the subject (first, second or any other subject) or a distance between markers placed on the skin of the subject. Li may therefore, be used to characterise the subject or to be used for calculating or determining other values. Li may be determined using different methods including finding the distance in image space between the pupils or by bounding the eyes with a polygon (e.g. rectangle) and finding the distance between the centres of those polygons, for example.

Optionally, determining the physical arrangement in space of the subject may further comprise the steps of:

determining a distance, z, between the first subject and the camera according to:

z=a+b(Li/L)

where L is the distance in real space (i.e. Euclidian distance) between the facial features of the first subject or between the markers placed on the skin of the first subject, a is a constant and b is a scaling factor. The eye reference point may be a point between the first subject's eyes (e.g. at a midpoint), for example.

Optionally, the camera may be a camera or a web camera connected to a computer or a camera forming part of a mobile device. The mobile device may be a mobile telephone, or mobile computer, for example.

Optionally, the physical arrangement in space of the subject (first, second or any other subject) may include any one or more of: position of the subject, location of the subject, orientation of the subject, a part of the subject, the eye or eyes of the subject or a point relative to the subject or a part of the subject.

Preferably, the subject may be a person. The subject may also be an animal.

Preferably, the eye measurement may include information or data that varies with the gaze of the first subject.

Preferably, the visual target may be presented on a screen. The screen may be a computer screen, for example.

Optionally, the eye measurement of the subject may be obtained using any one or more of: a camera, pupil tracking, electro-oculography, EOG, photo-oculography, POG, video-oculography, VOG, pupil centre cornea reflection, PCCR, infrared reflections from the eye, a pair of cameras, an infrared illuminator, Purkinje images, search coils and electrodes. Other techniques may be used.

According to a third aspect, there is provided an apparatus for tracking the gaze of a subject comprising:

a subject measuring device configured to capture a physical arrangement in space of a first subject and to obtain an eye measurement of the first subject;

a store of records each containing a physical arrangement in space of a second subject associated with data derived from one or more eye measurements obtained when the second subject was presented with the one or more visual targets; and

logic configured to:
match the captured physical arrangement in space of the first subject with a physical arrangement in space of a second subject in the store of records, and
provide an output indicating a gaze of the first subject based on the obtained eye measurement of the first subject and the data derived from the one or more eye measurements associated with the matched physical arrangement in space of the second subject.

Preferably, the apparatus may further comprise a visual target presenting device and wherein the logic is further configured to:
(a) present the second subject with a visual target;
(b) determine a physical arrangement in space of the second subject when presented with the calibration target;
(c) obtain an eye measurement of the second subject when presented with the visual target;
(d) store in the store of records a record of the second subject's physical arrangement in space and data derived from the eye measurement of the second subject; and
(e) repeating steps a) to d) for one or more further different visual targets.

Optionally, the logic may be further configured to repeat the steps of a) to e) for the same visual target and different physical arrangements in space of the second subject.

Optionally, the first subject and the second subject are the same or different individuals.

The methods described above may be implemented as a computer program comprising program instructions to operate a computer. The computer program may be stored on a computer-readable medium.

It should be noted that any feature described above may be used with any particular aspect or embodiment of the invention. In particular, any or all of the methods and systems (or individual features) described for calibrating may be implemented together with any or all of the methods and systems (or individual features) for tracking the gaze of a subject.

### BRIEF DESCRIPTION OF THE FIGURES

The present invention may be put into practice in a number of ways and embodiments will now be described by way of example only and with reference to the accompanying drawings, in which:

FIG. 1 shows a schematic diagram of an eye tracking system showing a set of calibration targets, given by way of example only;

FIG. 2 shows a flow chart of a method from calibrating the eye tracking system of FIG. 1;

FIG. 3 shows a flow chart of a method for tracking a subject's gaze using the system of FIG. 1, calibrated according to the method of FIG. 2;

FIG. 4 shows a schematic diagram of the subject of FIG. 3 indicating reference points on the subject's head; and

FIG. 5 shows a schematic diagram of the eye tracking system of FIG. 1 including a coordinate system used to locate the subject of FIG. 4.

It should be noted that the figures are illustrated for simplicity and are not necessarily drawn to scale.

### DETAILED DESCRIPTION OF THE PREFERRED EMBODIMENTS

Eye-Tracking Method

There are two main approaches to eye tracking. The first approach is to measure the position of the eye with respect to the head. Because eye-position is relative to head position, this technique is not able to determine point-of-regard or gaze unless the head position and pose is also tracked. In order to understand why measuring eye-movement relative to the head does not give point of regard information, consider a tracking subject looking at a point in space directly in front of him with his head in a neutral, forward facing position. In this situation the position of the iris/pupil is approximately in the centre of the ocular cavity. Now consider the tracking subject rotating his head to the left while remaining focused on the same point in space. The relative position of the iris/pupil is now toward the right of the ocular cavity. Although the eye has moved relative to the head, the point of regard is the same in both cases. For this reason, it is difficult to infer point of regard from relative eye-movements.

The second approach is to measure the orientation of the pupil with respect to a Purkinje image produced by an infra-red illuminator.

The list below, gives an overview of four categories of eye-tracking technologies:

Electro-OculoGraphy (EOG)—this technique involves attaching electrodes to the skin surrounding the eye so as to measure the electric potential differences from which the orientation of the eye can be determined. However, because this technique estimates eye movement relative to the head, it cannot be used for point-of-regard determinations unless the head position and pose is also tracked.

Scleral contact lens/search coil—this technique involves the use of a contact lens with an attached search coil. The movement of the search coil through an electromagnetic field is recorded from which the movement of the eye relative to the head can be deduced. The main advantage of this approach is that it is more accurate than other eye-tracking technologies. The main disadvantages of this approach is that the user must wear the scleral search coil and, as eye movement is measured relative to the head, point of regard determinations are only possible if the technique is combined with head-tracking.

Photo-OculoGraphy (POG)—this is a broad grouping of eye-tracking techniques that detect and measure features of the eye such as the location of the pupil or iris, track the boundary between the iris and sclera (limbus tracking) or detect and track a Purkinje image (infra-red corneal reflection). Although, the techniques in this category are many and varied, perhaps the most popular technique is ‘limbus tracking’ which is the process of tracking the boundary of the iris and sclera. Limbus tracking usually involves the use of a head-mounted device consisting of photodiodes and the use of an infra-red illuminator. However, other limbus tracking techniques are known and usually require the head to be fixed with a chin rest or bite bar. Techniques within this category generally do not provide point of regard information unless the tracking subject's head is fixed in space or the head position and pose is tracked so as to distinguish between the relative movement of the eye based upon eye movements and the relative movement of the eye based upon head movements (such as head rotation).

Video-OculoGraphy (VOG)—this is a grouping of eye-tracking techniques that are based upon detecting the centre of the pupil and the centre of the Purkinje image (also known as corneal reflection or glint) from a light source within the frames of a video of the tracking subject's eye. Typically, an infra-red light source is used to produce the Purkinje image. The advantage of recording the centre of the Purkinje image is that its position is relatively constant under moderate head movements. As the location of the Purkinje image is relatively constant, the relative position of the pupil centre to the centre of the Purkinje image can be used to normalise the pupil centre. What this means is that if the normalised pupil centre depends upon the position of the eye in the ocular cavity and is invariant to moderate changes in head pose. A calibration procedure is used to map the normalised pupil centre onto screen co-ordinates. Typical calibration procedures involve getting the tracking subject to look at a moving token as it traverses the screen. The relative position of the pupil centre to the centre of the Purkinje image is recorded as the calibration token rests at known screen co-ordinates. An interpolative mathematic approach is then used to map the normalised pupil centre onto screen co-ordinates. Interpolation may be used especially with Pupil Centre Corneal Reflection eye-tracking techniques.

Most commercial eye-tracking systems use the VOG approach for remote, non-invasive eye tracking. The VOG method, itself, can be classified into two groups: those based on interpolation based methods [1], [2], [3], [4] and those based on model based methods [5], [6], [7], [8], [9], [10], [11]. Interpolation based methods map image-based eye features onto gaze points. Model based methods construct a geometrical model of the features of the eye and use this model to estimate the gaze vector. Overall, interpolative methods are more common in commercial eye-tracking systems because they have simpler requirements and, for this reason, tend to be more robust. Although, model based methods are more complex to set up, they tend to offer a greater degree of freedom of head movement.

The following sections will discuss the 2D interpolation-based gaze estimation technique and the 3D model-based gaze estimation technique.

2D Interpolation-Based Gaze Estimation Technique

The 2D interpolation based gaze estimation technique uses a system calibration procedure to map 2D eye movements in a video image of the tracking subject's eye onto screen co-ordinates without actually calculating the eye-gaze trajectory. The Pupil Centre Cornea Reflection (PCCR) technique is the most commonly used 2D mapping-based approach for eye gaze tracking [12], [13], [14], [15]. The mapping function used in the calibration process is typically based upon a bilinear interpolation of the pupil centre and the centre of a Purkinje image (infra-red reflection from the surface of the cornea). Because the co-ordinates of the centre of the Purkinje image are relatively invariant to small head movements, the relative position of the pupil centre to the Purkinje image at known screen focal points can be used to build the eye-gaze to screen mapping function. Unfortunately, the co-ordinates of the pupil centre and centre of the Purkinje image also vary significantly with moderate head movements making the 2D interpolative gaze estimation technique very sensitive to head motion [16]. For this reason, the tracking subject has to keep his/her head unnaturally still in order to achieve good performance.

Systems built from the PCCR technique can show high accuracy if the tracking subject does not move their head. If the head position is fixed with a chin-rest or bite bar then tracking error can be less than one degree in the visual angle. This corresponds to an error of about 1 cm on a display screen if the tracking subject's eye is at a distance of 60 cm from the display. However, the accuracy of the gaze estimation is known to drop dramatically when the tracking subject moves his/her head. Changes in depth can be particularly problematic [16], [13]. Typically the loss in accuracy will necessitate a recalibration for all but the most minor head movements. The need for repeated recalibration has a large impact on the usability of these eye-tracking systems.

3D Model-Based Gaze Estimation Technique

Model based methods use geometric models of the eye to estimate the eye-gaze vector [16], [17], [18], [19]. The eye model is usually constructed from the centres and radii of the eyeball and cornea; the position of the foveola, the central region of the fovea; the centre of the pupil; the optical axis of the eye defined by the centres of the eyeball, cornea, and pupil; and the visual axis of the eye, defined by the line connecting the foveola and the point of regard, that also passes through the centre of corneal curvature. Most model based methods rely on stereo cameras [19], [20] although single camera solutions have also been suggested in [11], [17]. In both cases, the cameras need to be calibrated and the scene geometry must be known so that the point of regard or gaze can be calculated.

Some work has been done to overcome the need for system and user calibration [21]. They use a corneal reflection technique which aims to create an eye tracking system that would, in theory, allow freedom of head movement, while not requiring any kind of system or user calibration. Unfortunately, the simplifying assumptions made by basic corneal reflection technique have a big impact upon its accuracy. Other researchers have worked to improve the head motion tolerance of eye-tracking algorithms [22], [23], [24], [25] though these methods are still sensitive to head motion.

Eye-Tracking Systems

Most of the commercially available eye gaze tracking systems [26], [27], [28] [29] are built on the PCCR technique. These commercial systems claim that they can tolerate small head movements. For example, LC technologies [26] have an eye-tracker than can accommodate head motion of the order of less than 6.5 cm² (2 square inches). The ASL eye tracker [27] has the best claimed tolerance of head movement, allowing approximately 930 cm² (one square foot) of head movement. It eliminates the need for head restraint by combining a magnetic head tracker with a pan-tilt camera. However, details about how it handles head motion are not publicly known. Furthermore, combining a magnetic head tracker with a pan-tilt camera is complicated and requires camera and system calibration and is complex and expensive for the regular user.

In summary, the system and method of the present disclosure may use or incorporate any of the above-described techniques. However, existing implementations of eye tracking systems based on the PCCR technique, in particular, share two common drawbacks: first, the user has to perform certain calibration processes to establish the relationship between the on-screen calibration points and the user-dependent eye parameters before using the gaze tracking system; second, the user has to keep his head unnaturally still, with no significant head movement allowed.

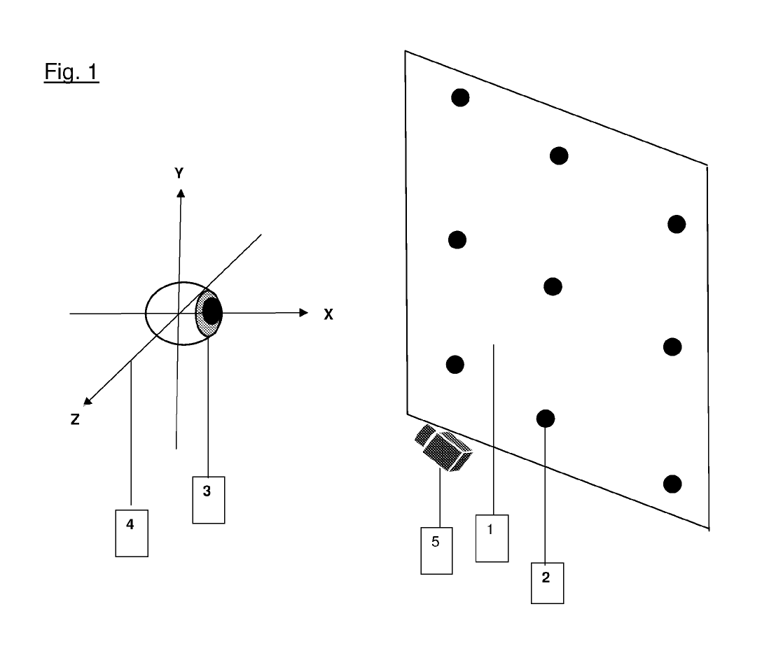

One embodiment of the eye-tracker system is illustrated in FIG. 1. This figure shows a surface 1 upon which are distributed one or more calibration target positions 2. One eye or both eyes 3 of a human or animal calibration subject are located in Cartesian space defined by co-ordinate axes (X, Y, Z) 4. Eye-gaze measurements or eye images are recorded by the eye-tracking device with a camera 5 as the calibration subject looks at each calibration target 2 in turn. These eye-gaze measurements or eye images are stored together with the surface 1 co-ordinates of the calibration target 2 to which they relate and the physical arrangement in space of the subject as recorded in the camera 5 image. The physical arrangement of the subject may include any one or more of position and/or orientation and/or scale of the eye or eyes (or a reference point related to the eye or eyes). The physical arrangement in space may also include information that may be used to derive or describe the location, position and/or orientation of the subject or a portion of the subject, for example. The process may be repeated for all (or more than one) calibration targets 2 on the surface 1. The calibration targets 2 may be displayed separately (e.g. in turn). A calibration procedure or routine may then be performed to determine the mapping of the calibration subject's eye-gaze (estimated or calculated from the stored eye measurements or eye images) onto the display for the given subject physical arrangement in space. The calibration data is then stored in the eye-tracking device memory or on a storage device together with the physical arrangement in space of the subject for which the eye-tracker system was calibrated. The process may then be repeated for a number of different eye or eyes (or eye or eyes reference point) physical arrangements in space.

During normal operation of the eye-tracker apparatus, it is possible to build up a repository of calibration data for a number eye or eyes (or eye or eyes reference point) for different physical arrangements in space, by storing the calibration subject's eye or eyes (or eye or eyes reference point) physical arrangements in space together with the calibration data whenever the user performs calibration. This has the effect of pre-computing a range of eye-tracker calibrations for different physical arrangements in space of the subject, eye or eyes (or a reference point related to the eye or eyes).

When the eye-tracker uses the PCCR eye-tracking technique in particular, a typical eye measurement may record the co-ordinates of the pupil centre and the centres of one or more Purkinje images (glints) produced on the surface of the cornea by one or more infra-red illuminators. These eye measurements would be taken as the calibration subject looks at each calibration target 2 on the surface 1.

When building up a repository of calibration data for different subjects, eye or eyes (or eye or eyes reference points), i.e. for different physical arrangements in space, the stored calibration data may be specific to the position and/or orientation of the camera 5, infra-red illuminators (if the eye-tracker uses the PCCR technique) and surface 1 and/or optical properties of the camera 5. These data may be absolute data or relative to the arrangement of the camera 5, for example. Therefore, it is possible to store the position and/or orientation of the camera 5, infra-red illuminators and surface 1 and/or optical properties of the camera 5 for each calibration. In this way, the eye-tracker system can build up the repository of calibration data to be reused in the future whenever the physical arrangement in space of the tracking subject's eye or eyes (or eye or eyes reference point) matches one of the physical arrangement in space of the of a calibration subject (or their eye or eyes) stored by the eye-tracker for a given position and/or orientation of the camera 5, infra-red illuminators and surface 1 and optical properties of the camera 5 for which they are stored.

The following provides and example for creating a calibration store.

A calibration subject may be asked to sit in one physical arrangement and look at visual targets, for example, nine dots on a screen arranged in 3 rows of 3 columns (e.g. top left, top middle, top right, middle left, middle middle, etc.) While looking at each visual target the calibration subject is asked to remain still and only move her eyes when looking at each visual target. Her physical arrangement is recorded or captured as she looks at visual target 1, the location of visual target 1 is recorded, and one or more eye measurements are also recorded (this may depends upon how long the calibration subject looks at visual target but typically it may be for a couple of seconds). Several eye measurements may be recorded (e.g. maybe 50) for each visual target because eye measurements may be inaccurate. Obtaining more readings may improve reliability and reduce errors.

This procedure is repeated for all visual targets. Furthermore this may be repeated for several physical arrangements.

An example calibration store may take the following form:
Physical arrangement 1
   Visual Target 1
      Eye Measurement 1
      Eye Measurement 2
      Eye Measurement 3
      :
      Eye Measurement n
   Visual Target 2
      Eye Measurement 1
      Eye Measurement 2
      Eye Measurement 3
      :
      Eye Measurement n
   Visual Target 3
      Eye Measurement 1
      Eye Measurement 2
      Eye Measurement 3
      :
      Eye Measurement n
   Visual Target n (n tends to be 9, 16, 24)
      Eye Measurement 1
      Eye Measurement 2
      Eye Measurement 3
      :
      Eye Measurement n (n depends upon how many samples we take)
Physical arrangement 2
   Visual Target 1
      Eye Measurement 1
      Eye Measurement 2
      Eye Measurement 3
      :
      Eye Measurement n
   Visual Target 2
      Eye Measurement 1
      Eye Measurement 2
      Eye Measurement 3
      :
      Eye Measurement n
   Visual Target 3
      Eye Measurement 1
      Eye Measurement 2
      Eye Measurement 3
      :
      Eye Measurement n
   Visual Target n
      Eye Measurement 1
      Eye Measurement 2
      Eye Measurement 3
      :

Eye Measurement n
Physical arrangement n (n may be several thousand)
   Visual Target 1
      Eye Measurement 1
      Eye Measurement 2
      Eye Measurement 3
      :
      Eye Measurement n
   Visual Target 2
      Eye Measurement 1
      Eye Measurement 2
      Eye Measurement 3
      :
      Eye Measurement n
   Visual Target 3
      Eye Measurement 1
      Eye Measurement 2
      Eye Measurement 3
      :
      Eye Measurement n
   Visual Target n
      Eye Measurement 1
      Eye Measurement 2
      Eye Measurement 3
      :
      Eye Measurement n

Alternative data structures may be used. It is noted that when a calibration subject is asked to sit still while looking at the visual targets they may move a little (people find it difficult to remain perfectly still). Therefore, records may be based upon approximately the same physical arrangement.

Once a calibration store is built up these data may be further analysed and developed. This may be offline, during which time records for each physical arrangement may be analysed to generate coefficients of a polynomial interpolation function. Therefore, the data in the calibration store may be a sequence of coefficients (e.g. six) or it may be possible to calibrate online to work out the coefficients as and when they are required.

As an example, in order to track a subject presented to an eye-tracker apparatus, the following procedure may be carried out.

The physical arrangement of the tracking subject may be obtained.

1) when interpolation is to be used then we may:
a) Retrieve the records for the closest matching physical arrangement and work out the coefficients of the polynomial interpolation function for that physical arrangement. Alternatively, if calibration has occurred offline then the coefficients may be retrieved directly from the calibration store.
b) Eye measurements of the tracking subject are obtained and fed as an input into the interpolation function, which gives screen coordinates of eye gaze as an output.

2) if we are not doing interpolation then:
a) Retrieve the records for the closest matching physical arrangement AND the closest matching eye measurement. If there is a match then it may be assumed that the tracking subject is looking at the screen location for which the calibration subject was looking when the stored eye measurement was taken.

The distinction between 1) interpolation and 2) matching eye measurements may also be based on the nature of the eye measurements. For example, interpolation may be used when using an infra-red camera and an infra-red illuminator. Matching eye measurements may be used especially for coarse eye measurements that are taken with other camera types. This may involve obtaining templates or images of the eye.

Once the calibration procedure has been completed, the eye-tracker is now calibrated for a range of subject physical arrangements in space, eye or eyes (or eye or eyes reference point) positions and/or orientations and/or scales stored during calibration for a given position and/or orientation of the camera 5, infra-red illuminators and surface 1 and the optical properties of the camera 5. Should the calibration subject or a different tracking subject move such that if their physical arrangement in space changes then the current physical arrangement in space of the calibration or tracking subject's eye or eyes (or eye or eyes reference point) can be estimated within the image or video taken from the eye-tracker camera 5. It would then be possible to select the saved calibration data corresponding to the best match between the tracking subjects' physical arrangement in space with the stored physical arrangements in space stored by the eye-tracker during calibration.

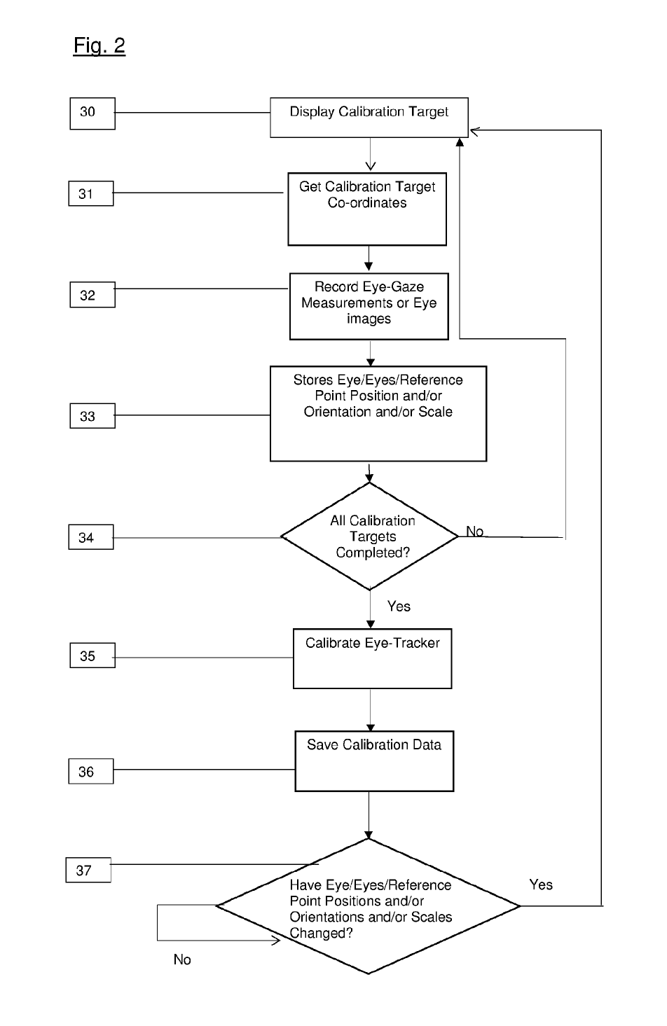

FIG. 2 shows a flowchart that outlines the eye-tracker operation. The flowchart in FIG. 2 will be explained with reference to FIG. 1. The process begins by getting the calibration subject to look at one calibration target 2 (FIG. 1) on a surface 1 (FIG. 1) in process 30. The eye-tracker stores the co-ordinates of the calibration target 2 (FIG. 1) as it is displayed on the surface 1 (FIG. 1) in process 31. The next step is to record eye measurements (such as the position of the pupil and Purkinje images from one or more infra-red illuminators) or eye images as the calibration subject looks at the displayed calibration target 2 (FIG. 1) in process 32. Process 33 stores the physical arrangement in space of the subject (e.g. of their eye or eyes or a reference point associated with the subject). The eye-tracker will then check that all the calibration targets 2 (FIG. 1) have been displayed in process 34. If there are more calibration targets 2 (FIG. 1) to be displayed then process 34 branches to process 30 to display the next calibration target 2 (FIG. 1). If all calibration targets 2 (FIG. 1) have been displayed then process 34 branches to process 35 to calibrate the eye-tracker. Process 36 then stores the eye-tracker calibration data. Process 37 tests whether the position subject's physical arrangement in space has changed and braches to process 30 if this is true otherwise process 37 loops back on itself until all physical arrangements in space of the subject have been calibrated.

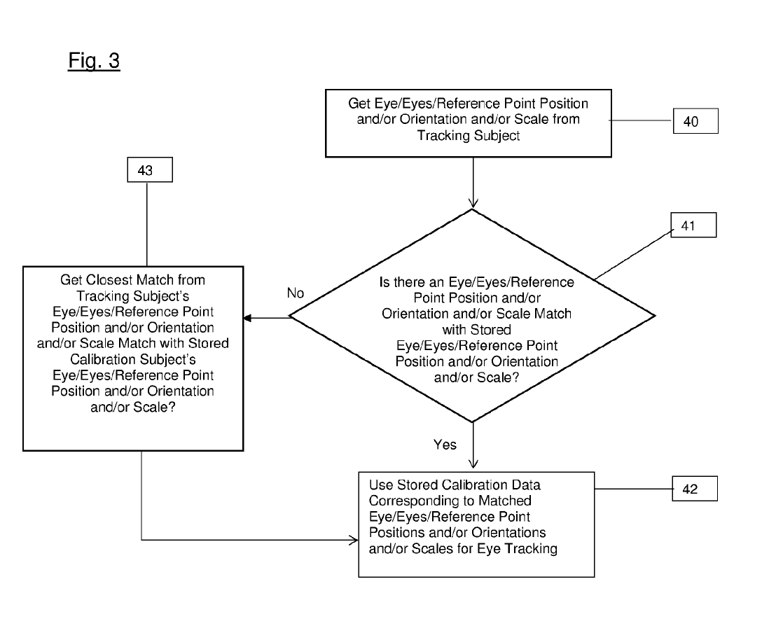

FIG. 3 gives a flowchart for the procedure of selecting stored calibration data for a given physical arrangement in space of the tracking subject once the eye-tracker device has been calibrated as outlined in FIG. 2. Process 40 captures the physical arrangement in space of the subject. Process 41 searches for a stored physical arrangement in space of the calibration subject, e.g. their eye or eyes (or eye or eyes reference point), that matches the current physical arrangement in space of the subject. If there is a match then process 42 extracts the stored calibration data for the matching physical arrangement in space of the subject. This calibration data is then used for eye-tracking. If there is no match then process 41 branches to process 43 that searches for the closest match between the tracking subject's physical arrangement in space and that of the stored calibration subject as stored during calibration. There are a number of possible matching strategies that can be used in process 43 such as finding the minimum Euclidean distance between the tracking subject's eye or eyes (or eye or eyes reference point) position and the stored calibration subject's eye or eyes (or eye or eyes reference point) positions. Once the closest match is found then process 43 branches to process 42. Alternatively, if a match between current and calibrated positions and/or orientations and/or scales cannot be found, the calibration data may be interpolated from the calibration data of two or more closest calibrated positions and/or orientations and/or scales, for example.

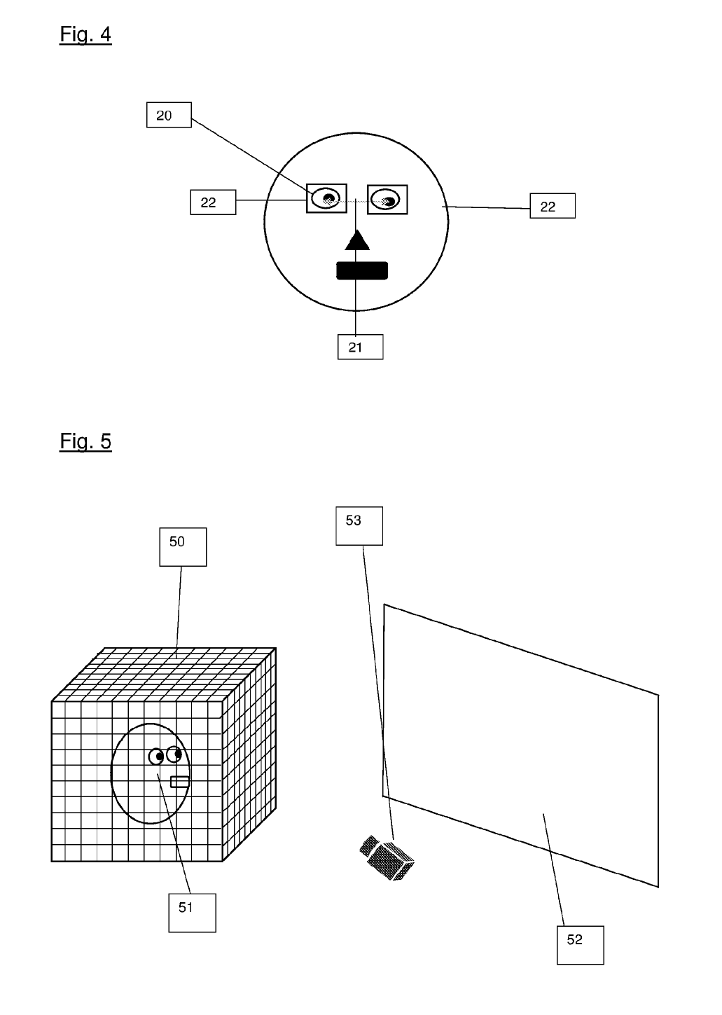

The overall procedure in FIG. 4 operates whenever the physical arrangement in space of a subject changes or changes more than some threshold value.

### First Embodiment

In the first embodiment, the method of estimation the physical arrangement in space of the subject is illustrated in FIG. 4. The positions of both the calibration subject's eyes 20 are measured with respect to an eye reference point 21 defined as the midpoint of the line joining the centre of the bounding rectangle 22 of each eye in the video image. The depth of the tracking subject's eyes is estimated by finding the Euclidean distance between the centre of a bounding rectangle 22 of each eye in the video image and dividing this length (measured in image-based pixel co-ordinates) by the Euclidean distance between the centre of the bounding rectangles of the tracking subject's eyes measured on the tracking subjects actual face (measured in the real-world co-ordinate system). The distance of the eye reference point from the camera z was then estimated using the perspective projection (Equation 1) below:

z=a+b*(Li/L) (1)

where Li is the distance between the subject's eyes in images space. For example, this may be the Euclidean distance between the centres of bounding rectangles of the eyes in image space. L is the distance between the subject's eyes in real space. For example, this may be the Euclidean distance between the centres of the bounding rectangles of the eyes in real-world units. a is a constant an b is a scaling factor linked to the camera focal length.

When the eye-tracker uses the PCCR eye-tracking technique, if the eye or eyes (or eye or eyes reference point) of different tracking subjects are located in the same position and/or orientation and/or scale and the direction of gaze is the same, the coordinates of the pupil centres and centres of the infra-red glints will be approximately the same for different tracking subjects. This means that if the eye-tracker is calibrated for one or more subject physical arrangement in space then these calibrations can be stored and reused at a later time by any tracking subject with the same or similar physical arrangement in space, i.e. who is positioned such that his/her eye or eyes (or eye or eyes reference point) are located at the same position and/or orientation and/or scale as one of the pre-calibrated positions and/or orientations and/or scales.

In order to calibrate the eye-tracker device for different positions of the eye reference point 21 (FIG. 4), the co-ordinates of a 3D tracking box 50 (FIG. 5) centred on the tracking subjects head 51 (FIG. 5) when the tracking subject is sitting in a natural position may be estimated where the dimensions of the tracking box 50 (FIG. 5) are large enough to capture the full range of natural head movements for a tracking subject looking at the surface 52 (FIG. 5). The eye-tracker device camera 53 (FIG. 5) may be positioned such that it can capture images of the tracking subject's head 51 (FIG. 5) as it moves within the tracking box 50 (FIG. 5). For instance, when the surface 52 (FIG. 5) is a computer monitor, a typical tracking box 50 (FIG. 5) might be a box of 60 cm width, 60 cm height and 60 cm depth. However, the tracking box 50 (FIG. 5) could be of other dimensions. The tracking box 50 (FIG. 5) could then be divided into uniform or non-uniform cells. The number of cells used may then depend upon the degree of accuracy required for the eye-tracker device. For instance, the tracking box could be divided into 1000 uniform cells by dividing the width, height and depth by 10 such that there are 10*10*10 cells. The more cells, the more accurate eye-tracker device would be when fully calibrated because the eye-tracker device could store more calibrations for different subject physical arrangements in space.

To calibrate the eye-tracker device, the calibration subject may move such that the eye-reference point 21 (FIG. 4) may be located in the centre of each cell of the tracking box 50 (FIG. 5). The calibration procedure outlined in the flowchart in FIG. 2 may then be performed to create and store calibration data for each cell of the tracking box 50 (FIG. 5). However, process 37 (FIG. 2) completes when eye reference point 21 (FIG. 4) is located in each cell of the tracking box and the eye-tracker has been calibrated.

Once the eye-tracker device has been calibrated, the eye-tracker device (or a processor or logic operating in conjunction with the eye-tracker device) may estimate the current position of the eye-reference point 21 (FIG. 4) of the tracking subject in Cartesian co-ordinates to determine the cell of the tracking box that contains the eye reference point 21 (FIG. 4). The eye-tracker (or processor, not shown) will then retrieve the stored calibration data from the eye-tracker device memory or a storage device and use this calibration data for eye-tracking.

### Second Embodiment

Another embodiment of the eye-tracking system uses a web-camera attached to a desktop computer or mobile device (e.g. Ea cellphone, smart phone or tablet computer). The camera captures a video of the user's face while they use a software application such as a web browser. The eye-tracking device detects the users' eyes in the video image and captures images of one or both eyes or makes eye-gaze measurement as the eyes look at one or more known calibration targets 2 (FIG. 1). A calibration target 2 (FIG. 1), could be a displayed token which the user looks at on the computer or mobile device display or the calibration target could be the co-ordinates of an on-screen selection using a touch screen or an input device such as a mouse or stylus. The eye tracker device may store eye-gaze measurements or images of the eye as it looks at known calibration targets 2 (FIG. 1) and store the corresponding physical arrangement in space of the subject, e.g. their position and/or orientation and/or scale of the eye or eyes (or eye or eyes reference point). The position could be measured as the position of an eye in the camera image or the position of an eye reference point (such as the centre of the bounding rectangle of the eye) in pixel co-ordinates and/or estimating depth of the eye or reference point using the perspective projection based upon the relative scale of the eye or any other feature that allows depth to be estimated. In this way, the on-screen co-ordinates of the calibration target 2 (FIG. 1) together with the position and/or orientation and/or scale of the eye or eyes (or eye or eyes reference point) and eye-gaze measurements and/or eye images are stored in the device memory or on a storage device (local or remote). This process may be repeated for more than one calibration points and for different eye or eyes (or eye or eyes reference point) positions and/or orientations and/or scales so as to build a repository of calibration data.

When the tracking subject's physical arrangement in space (such as the position of their eye or eyes, or eye or eyes reference point) moves into the vicinity, close to or within a predetermined limit or tolerance of a stored calibrated physical arrangement, position and/or orientation and/or scale, the eye tracker will compare the stored eye-gaze measurements or stored eye images with the current eye-gaze measurements and/or eye images of the tracking subject. The degree of correspondence between the calculated and stored eye or subject measurements and/or eye images will be determined and a match above a given threshold will be taken to indicate that the user is looking in the vicinity of the display co-ordinates of the stored calibration target 2 (FIG. 1) for which the stored physical arrangement in space, eye or eyes (or eye or eyes reference point) position or/or orientation and/or scale were stored. In this way calibration data for a set of eye positions and/or orientations and/or scales are stored in device memory or on a storage device so that they can be recalled for future use.

### Third Embodiment

Another embodiment may use a Template Matching algorithm where templates of the calibration subject's eye or eyes are captured as the calibration subject looks at one or more calibration targets 2 (FIG. 1) on a surface 1 (FIG. 1). When capturing the eye templates, the templates are stored together with the position and/or orientation and/or scale of the calibration subject's eye or eyes (or eye or eyes reference point) and co-ordinates of the calibration target 2 (FIG. 1) on the surface 1 (FIG. 1). This means that if the eye-tracker stores eye templates for one or more eye or eyes (or eye or eyes reference point) positions and/or orientations and/or scales then these templates can be stored and reused at a later time by any tracking subject who is positioned such that his/her eye or eyes (or eye or eyes reference point) are located at the same position, orientation and scale as one of the pre-calibrated positions, orientations and scales stored by the eye-tracking device during calibration. When the tracking subject's eye or eyes (or eye or eyes reference point) are located at or close to (e.g. within a predetermined distance) a pre-calibrated position, orientation and scale then the stored eye-templates can be matched with the tracking subjects eye or eyes and a match above a threshold level can be used to signify that the tracking subject is looking in the vicinity or direction of the position of the calibration target on the surface for which the eye template or templates where captured.

Head tracking or determining a physical arrangement in space of the subject may be used to build up multiple calibrations. For example, the subject may be above or below a display screen. The subject may have their physical arrangement in space determined (for example, using a camera). The method may be used multiple times to generate calibrating data for different positions or orientations of the subject. The subject may be moved and calibrated repeatedly, for example.

The ‘physical arrangements’ and their calibrations may be stored. If a new subject interacts with the system then their physical arrangement may be compared and matched with those stored in the eye-tracker. The corresponding calibration may then be retrieved (i.e. one or more visual targets associated with the stored physical arrangement). Having done this, we can now track the gaze of the new subject.

Instead of storing the calibration with each physical arrangement, we may store the eye-measurements and calibration targets that we use to calibrate. This allows performance of calibration in real-time.

The head tracking (i.e. determining a physical arrangement in space of a subject) may be done in many ways. For example, the head may be tracked using camera or video techniques. Physical sensors may be attached to the head that specify location and orientation of the head. Other techniques or methods may be used.

Storing multiple calibrations for different head positions (i.e. physical arrangements in space) allows the selection or matching of an optimum calibration set based on the determined physical arrangement in space of the current subject. This reduces the need to calibrate for each user. Building up a store of many calibrations and tracking head or other physical arrangements to select the correct calibration, enables a calibration set to be used for multiple users

In example implementations, the system may store:

1) The physical arrangement of the tracking subject;

2) The eye measurements when the tracking subject is looking at a calibration target; and

3) The location of the calibration target.

The eye-tracker may typically store several eye-measurements for several calibration targets for any one position in space.

A match between the current physical arrangement of the tracking subject and eye-measurement indicates that the tracking subject is looking at the point in space indicated by the location of the calibration target for which the stored eye measurement relates.

However, the following may also be true:

If there is a match between the current physical arrangement of the tracking subject and the stored physical arrangement but there is no match between the current eye-measurements and the stored eye measurements then the stored eye measurements and their associated calibration targets may be retrieved to calibrate the system. This may be achieved using a mathematical calibration procedure such as bi-linear interpolation. This calibration procedure allows the eye-tracker to estimate the calibration subject's point of regard based upon his/her eye measurements even when there is no match in the store for his/her eye measurements.

In an example case, there may be nine calibration targets on a display screen arranged in a 3*3 grid. The tracking subject's physical arrangement may be captured as well as eye measurements when he/she is looking at each calibration target. The location of each calibration target is also stored or captured. This may be repeated for a plurality of physical arrangements.

If a different subject uses the system or is investigated by it then a closest matching physical arrangement in the store may be found to this new subject. Because the eye measurements were recorded for only nine calibration targets, it is unlikely that a match between current eye measurements and the stored eye measurements will be found. In this situation (which may be typical), the nine sets of eye measurements and nine calibration targets may be extracted from the store and a mathematical interpolation procedure may then be used to estimate the new tracking subject's point of regard on a screen (for example) based upon his/her current eye measurements using a polynomial interpolation procedure based upon the stored eye measurements and calibration targets.

When creating the store of calibration subject's physical arrangement and their eye measurements and associated calibration targets, then his/her interactions may be used as calibration targets.

For example, a subject may have a mobile device with a web camera. The mobile device may have software that tracks the physical arrangement from the camera image and tracks user interactions (e.g. with screen displayed objects) to use as calibration targets. When the user looks at an on-screen control and makes a selection, it is possible to take eye measurements of the subject using the camera and record the interaction point as a calibration target. Eye measurements may simply be images (templates) of the user's eyes as he/she looks at the control (calibration target). However, other eye measurements may be possible.

When using a mobile device with a web camera, the eye measurements may be captured (for example, as eye templates or images of the eye or eyes) when the tracking subject makes an on screen selection. For example, when a subject uses a tablet computer (e.g. an iPad) they may look at an on screen button and then tap it with their finger. An image of the subject's eye or eyes (viewing a particular button) may be captured immediately before or during the finger tap. The point of gaze may be captured or estimated as the centre of the button. In this way, the screen position of the button may be used as the visual or calibration target. A match may then be found between the subject's physical arrangement and a physical arrangement stored in the eye-tracker system.

In this example and other similar implementations (perhaps using other devices), a store of physical arrangements, eye measurements and calibration targets may be built up from one or more subjects. This store may be used to:

a) Match the user's current physical arrangement and eye measurement with those in the store to estimate gaze;

b) Match the user's current physical arrangement with a physical arrangement in the store. Retrieve the stored eye measurements and calibration targets. Do interpolation based upon the stored eye measurements and calibration targets and the user's current eye measurement to estimate gaze.

The store may hold different types of data. These may include:

a. The tracking subject's physical arrangement;

b. The eye measurements taken when the tracking subject is looking at a calibration target (there may be many readings for one calibration target); and

c. The location of the calibration target (e.g. its location on a screen).

Therefore, if a new subject uses the system then it is possible to find the closest matching physical arrangement in the store. It may be possible to find an exact or closely matching eye measurement (if one exists). If a matching eye measurement exists, it may be assumed that the new subject is looking at the calibration target for which the stored eye measurement was previously recorded.

However, usually there may be no exact or even close match between the user's current eye measurement and the eye measurements in the store. In this case, when a subject is presented to the system, the closest matching physical arrangement in the store may be found. This may still be classified as a match but further refinement may be required. The associated stored calibration targets may be retrieved together with their stored eye measurements for this physical arrangement.

The retrieved calibration targets and eye measurements may then be used to calibrate the eye-tracker using a mathematical polynomial interpolation function (for example, a mathematical procedure that may be bi-linear interpolation). This interpolation function may then be used to estimate the subject's point of regard even when there is no exact matching eye measurement in the store.

The polynomial interpolation function may be stored instead of the eye measurements and calibration targets. The eye measurements and calibration targets may be calibrated dynamically. For example, the interpolation function may be stored if it is calculated offline or we can calibrate using the eye measurements and calibration targets online as and when required.

### REFERENCES

[1] Z. Zhu and Q. Ji, Eye and gaze tracking for interactive graphic display, Machine Vision and Applications, vol. 15, no. 3, pp. 139-148, 2004.

[2] K. H. Tan, D. Kriegman, and H. Ahuja, Appearance based eye gaze estimation, in Proceedings of the IEEE Workshop on Applications of Computer Vision, 2002, pp. 191-195.

[3] J. Zhu and J. Yang, Subpixel eye gaze tracking, in Proceedings of the IEEE International Conference on Automatic Face and Gesture Recognition, Washington D.C., 2002, pp. 131-136.

[4] C. H. Morimoto and M. Mimica, Eye gaze tracking techniques for interactive applications, Computer Vision and Image Understanding, Special Issue on Eye Detection and Tracking, 98(1), pp. 4-24, 2005.

[5] D. Beymer and M. Flickner, Eye gaze tracking using an active stereo head, in Proceedings of the International Conference on Computer vision and Pattern Recognition, 2003.

[6] S. W. Shih and J. Liu, A novel approach to 3-D gaze tracking using stereo cameras, in IEEE Transactions on Syst. Man and Cybern., part B, 34, pp. 234-245, 2004.

[7] J. Wang, E. Sung, and R. Venkateswarlu, Eye gaze estimation from a single image of one eye, in Proceedings of International Conference on Computer Vision, 2003.

[8] T. Ohno, N. Mukawa, and A. Yoshikawa, Freegaze: A gaze tracking system for everyday gaze interaction, in Proceedings of the symposium on ETRA 2002, 2002.

[9] C. H. Morimoto, A. Amir, and M. Flickner, Detecting eye position and gaze from a single camera and 2 light sources, in Proceedings of the International Conference on Pattern Recognition, 2002.

[10] Y. Matsumoto, T. Ogasawara, and A. Zelinsky, Behavior recognition based on head pose and gaze direction measurement, in Proceedings of 2000 IEEE/RSJ International Conference on Intelligent Robots and Systems, 2000.

[11] E. D. Guestrin and M. Eizenman, General theory of remote gaze estimation using the pupil center and corneal reflections, IEEE Transactions on biomedical engineering, 53(6), pp. 1124-1133, 2006.

[12] T. E. Hutchinson, K. P. White Jr., K. C. Reichert, and L. A. Frey, Human-computer interaction using eye-gaze input, in IEEE Transactions on Systems, Man, and Cybernetics, 19, pp. 1527-1533, 1989.

[13] R. J. K. Jacob, Eye-movement-based human-computer interaction techniques: Towards non-command interfaces, 4, pp. 151-190, Ablex Publishing corporation, Norwood, N.J., 1993.

[14] Y. Ebisawa, M. Ohtani, and A. Sugioka, Proposal of a zoom and focus control method using an ultrasonic distance-meter for video-based eye-gaze detection under free-hand condition, in Proceedings of the 18th Annual International conference of the IEEE Eng. in Medicine and Biology Society, 1996.

[15] C. H. Morimoto, D. Koons, A. Amir, and M. Flickner, Pupil detection and tracking using multiple light sources, Image and Vision Computing, 18, pp. 331-336, 2000.

[16] C. H. Morimoto and M. Mimica, Eye gaze tracking techniques for interactive applications, Computer Vision and Image Understanding, Special Issue on Eye Detection and Tracking, 98(1), pp. 4-24, 2005.

[17] C. Hennessey, B. Noureddin, and .P Lawrence, A single camera eye-gaze tracking system with free head motion. In ETRA '06: Proceedings of the 2006 symposium on Eye tracking research & applications, pp. 87-94, New York, N.Y., USA., ACM Press, 2006.

[18] E. Guestrin and M. Eizenman, Remote point-of-gaze estimation requiring a single point, Proceedings of the 2008 symposium on Eye tracking research and applications., pp. 267-274, Savannah, March, 2008.

[19] D. Model and M. Eizenman, User-calibration-free remote gaze estimation system, in Proceedings of the 2010 Symposium on Eye-Tracking Research & Applications Austin, Tex., USA: ACM, pp. 29-36, 2010.

[20] T. Nagamatsu, J. Kamahara, T. Iko, and N. Tanaka, One-point calibration gaze tracking based on eyeball kinematics using stereo cameras. In Proceedings of the 2008 symposium on Eye tracking research & applications (ETRA '08). ACM, New York, N.Y., USA, pp. 95-98, 2008.

[21] D. Yoo , J. Kim, B. Lee, M. Chung, Non contact eye gaze tracking system by mapping of corneal reflections, Proc. Of the Int. Conf. on Automatic Face and Gesture Recognition, pp. 94-99, 2002.

[22] D.W. Hansen, Q. Ji, In the Eye of the Beholder: A Survey of Models for Eyes and Gaze, IEEE Transactions on Pattern Analysis and Machine Intelligence, 32(3), pp. 478-500, 2010.

[23] D. H. Yoo and M. J. Chung, A novel non-intrusive eye gaze estimation using cross-ratio under large head motion, Computer Vision and Image Understanding, Special Issue on Eye Detection and Tracking, 98, no. 1, pp. 25-51, 2005.

[24] F. L. Coutinho and C. H. Morimoto, Improving Head Movement Tolerance of Cross-Ratio Based Eye Trackers, International Journal of Computer Vision, 2012.

[25] J. J. Kang, E. D Guestrin, W. J. Maclean, and M. Eizenman, Simplifying the cross-ratios method of point-of-gaze estimation, In 30th Canadian medical and biological engineering conference (CMBEC30), 2007.

[26] “LC Technologies, Inc”, www.eyegaze.com.

[27] “Applied Science Laboratories”, www.a-s-l.com.

[28] “SensoMotoric”, www.smi.de.

[29] “Seeing Machines”, www.seeingmachines.com.

As will be appreciated by the skilled person, details of the above embodiment may be varied without departing from the scope of the present invention, as defined by the appended claims.

For example, other properties may be measured to determined or record the subject's physical arrangement in space. The targets may take any suitable shape or take any suitable form. For example, a target may move (and the test subject may follow it with eye measurements and the location of the target recorded, accordingly) or be stationary.

Many combinations, modifications, or alterations to the features of the above embodiments will be readily apparent to the skilled person and are intended to form part of the invention. Any of the features described specifically relating to one embodiment or example may be used in any other embodiment by making the appropriate changes.

1. A method for calibrating an eye tracker, comprising
(a) presenting a subject with a visual target;
(b) determining a physical arrangement in space of the subject;
(c) obtaining an eye measurement of the subject;
(d) storing a record of the subject's physical arrangement in space and data derived from the eye measurement of the subject, associated with the visual target presented to the subject; and
(e) repeating a) to d) for one or more further different visual targets.

2. The method of claim 1, further comprising repeating a) to e) for one or more of a different subject physical arrangement in space or the same physical arrangement in space.

3. (canceled)

4. The method of claim 1, wherein the data representing the subject's eye measurement is stored as a template representing the eye or eyes of the subject.

5. The method of claim 1 further comprising
generating parameters of a function having an output that describes the gaze of a subject when provided with an input representing an eye measurement of the subject.

6. The method of claim 5, wherein the data derived from the eye measurements are the parameters of the function.

7-9. (canceled)

10. The method of claim 5, wherein the function is a polynomial or a bi-linear function and the parameters are coefficients of the polynomial or bi-linear function.

11. A method for tracking the gaze of a subject, comprising:
(a) determining a physical arrangement in space of a first subject;
(b) obtaining an eye measurement of the first subject;
(c) matching the determined physical arrangement in space of the first subject with a physical arrangement in space of a second subject presented with one or more visual targets, wherein the physical arrangement in space of the second subject is associated with data derived from one or more eye measurements obtained when the second subject was presented with the one or more visual targets; and
(d) providing an output indicating a gaze of the first subject based on the obtained eye measurement of the first subject and the data derived from the one or more eye measurements of the second subject.

12. (canceled)

13. The method of claim 11, wherein a match between the determined physical arrangement in space of the first subject and the second subject occurs within a matching threshold.

14. The method of claim 11, wherein the data derived from the one or more eye measurements of the second subject are parameters of a function having an output that describes the gaze of a subject when provided with an input representing an eye measurement of the subject.

15-17. (canceled)

18. The method of claim 14, wherein the function is a polynomial or a bi-linear function and the parameters are coefficients of the polynomial or of the bi-linear function.

19. (canceled)

20. The method of claim 11, wherein matching further comprises determining a minimum Euclidian distance between the determined physical arrangement in space of the first subject and a physical arrangement in space of the second subject within any one or more stored records.

21. The method of claim 1, wherein the data derived from the one or more eye measurements of the second subject comprises a template representing the eyes of the second subject.

22. The method of claim 21, wherein providing the output further comprises:
matching the obtained eye measurement of the first subject with an eye measurement of the second subject obtained when the second subject was presented with a visual target; and
indicating the gaze of the first subject as the location of the visual target.

23. The method of claim 1, wherein determining the physical arrangement in space of the first subject further comprises determining an eye reference point from an image of the subject obtained by a camera.

24. (canceled)

25. The method of claim 11 further comprising determining a distance Li, which is a distance, as recorded in a camera image, between facial features of the first subject or a distance between markers placed on the skin of the subject.

26. The method of claim 25, wherein determining the physical arrangement in space of the subject further comprises:
determining a distance, z, between the first subject and the camera according to:
z=a+b(Li/L)
where L is the distance in real space between the facial features of the first subject or between the markers placed on the skin of the subject, a is a constant and b is a scaling factor.

27-31. (canceled)

32. The method of claim 1 wherein the eye measurement of the subject is obtained using any one or more of: a camera, pupil tracking, electro-oculography, EOG, photo-oculography, POG, video-oculography, VOG, pupil centre cornea reflection, PCCR, infrared reflections from the eye, a pair of cameras, an infrared illuminator, Purkinje images, search coils and electrodes.

33. An apparatus, comprising:
a subject measuring device configured to capture a physical arrangement in space of a first subject and to obtain an eye measurement of the first subject;
a store of records each containing a physical arrangement in space of a second subject associated with data derived from one or more eye measurements obtained when the second subject was presented with the one or more visual targets;
at least one processor; and
at least one memory storing computer executable instructions that, when executed by said at least one processor, further cause the apparatus to:
match the captured physical arrangement in space of the first subject with a physical arrangement in space of a second subject in the store of records, and
provide an output indicating a gaze of the first subject based on the obtained eye measurement of the first subject and the data derived from the one or more eye measurements associated with the matched physical arrangement in space of the second subject.

34. The apparatus of claim 33 further comprising a visual target presenting device and wherein the executable instructions, when executed by said at least one processor, further cause the apparatus to:
(a) present the second subject with a visual target;
(b) determine a physical arrangement in space of the second subject when presented with the calibration target;
(c) obtain an eye measurement of the second subject when presented with the visual target;
(d) store in the store of records a record of the second subject's physical arrangement in space and data derived from the eye measurement of the second subject; and
(e) repeating a) to d) for one or more further different visual targets.

35. The apparatus of claim 34, wherein the executable instructions, when executed by said at least one processor, further cause the apparatus logic to repeat a) to e) for the same visual target and different physical arrangements in space of the second subject.

36-39. (canceled)
